(19) United States
(12) Patent Application Publication
(10) Pub. No.: US 2003/0045930 A1
(43) Pub. Date: Mar. 6, 2003

(54) Apparatus and methods for packaging intrcorneal implants and facilitating placement thereof

(75) Inventor: Tuyet Hoc Nguyen, Orange, CA (US)

Correspondence Address: STOUT, UXA, BUYAN & MULLINS LLP, 4 VENTURE, SUITE 300, IRVINE, CA, 92618, US

(73) Assignee: Allergan Sales, Inc., Irvine, CA

(21) Appl. No.: 09/943,489

(22) Filed: Aug. 30, 2001

(51) Int. Cl. A61F 2/14

(52) U.S. Cl. 623/5.11; 606/107; 623/906

(57) ABSTRACT

Apparatus and methods for holding and packaging intracorneal implants are provided. In one embodiment, the apparatus includes a holder body having a surface sized and adapted to carry an intracorneal implant, and a through hole extending through the surface, preferably sized and adapted to facilitate suction being applied to the intracorneal implant located in proximity to the surface. A cap member preferably is provided and is sized and adapted to be secured to the holder body so as to hold an intracorneal implant between the surface of the body and the cap member. Methods for packaging intracorneal implants and for providing intracorneal implants ready for use are provided and may involve using such apparatus.

### BACKGROUND OF THE INVENTION

The present invention relates to apparatus and methods for packaging/holding intracorneal implants and facilitating placement of such implants in eyes. In particular, the invention relates to apparatus and methods for packaging or holding intracorneal implants ready for use, that is for implantation in or on a cornea, and to methods effective to facilitate such use of intracorneal implants.

As used herein, the term “intracorneal implant” refers to a structure, for example, a lens structure, which is sized, adapted and structured to be surgically attached in or on the cornea of an eye, for example, a mammalian eye, such as a human eye. As such, a “corneal implant” includes but is not limited to, corneal inlays, corneal onlays and the like. The present invention is particularly useful with corneal inlays.

The cornea comprises five layers, including an outer layer of epithelial cells. Bowman's membrane immediately posterior of the cells, the stroma immediately posterior of Bowman's membrane, Descemet's membrane immediately posterior of the stroma and the endothelium immediately posterior of Descemet's membrane. A number of surgical operations involve implanting a lens structure, for example, a corrective lens structure, into or onto one or more of these corneal components. For example, in one form of eye surgery, a portion or flap of corneal tissue is cut away exposing the stroma. A corneal inlay is placed on the stromal bed, and the corneal flap is put back on the cornea over the inlay. This procedure often does not require suturing. In another form of eye surgery, the layer of epithelial cells is removed and then a wedge-shaped annulus from Bowman's membrane and the underlying stroma are removed. An incision is then made from the posterior end of the resulting groove radially outwardly in an annual zone to define a flap. A corneal implant is attached by inserting the corneal implant beneath the corneal flap and fixing, e.g., suturing, it in place.

Hydrogel compositions have been suggested for use as materials of construction for corneal implants. As used herein, the term “hydrogel composition” refers to a composition including a material, for example, a polymeric material, and a sufficient amount of water to cause the composition to swell relative to the anhydrous material. Such hydrogel compositions often include at least about 38% by weight of water, and may include as much as about 60% or about 80% or about 90% or more by weight of water.

Corneal implants are quite fragile prior to implantation in the eye. For example, such implants can have diameters of about 0.1 inch or about 0.15 inch to about 0.2 inch or about 0.25 inch, and thicknesses of about 20 microns or less to about 50 or about 100 microns. Thus, excessive handling or even touching of the implants should be avoided. In addition, since the implants are thin and optically clear, they are often difficult to see with the naked eye. Moreover, corneal implants, for example, corneal inlays, should be placed in eyes so that the anterior face of the implant is placed anterior to the posterior face of the implant in the eye. Substantially reduced implant performance may occur if these faces are reversed or inverted in the eye. Thus, it would be advantageous to provide such implants so that implant handling is reduced, and the need for visual inspection of the implant is reduced.

Such implants should be packaged or otherwise provided so as to protect the implant and, in addition, facilitate the use of the implant during the surgical implantation procedure. Factors which must be considered in packaging/providing intracorneal implants include, but are not limited to, maintaining the integrity of the implant, particularly since the implant often is small, thin and hard to see, and includes a hydrogel and needs to be maintained in the hydrated state; reducing the amount of physical handling or touching of the fragile implant so as to reduce the risk of damaging the implant; reducing the amount of effort required to remove the implant from the packaging in order to simplify the surgical implantation and facilitate an advantageous outcome for the patient; and the like factors.

There is a need to provide new packaging/holding apparatus for intracorneal implants and methods to facilitate the effective use of such implants.

### SUMMARY OF THE INVENTION

New apparatus and methods for packaging/holding intracorneal implants and methods for packaging and providing such corneal implants for use have been discovered. The present apparatus are straightforward in construction, can be produced cost effectively and are highly effective and advantageous in use. For example, the present apparatus are effective in protecting intracorneal implants, particularly intracorneal implants, such as intracorneal inlays, which include hydrogel materials. In addition, the present invention provides intracorneal implants in packaging systems which can be easily opened so as to make the implants readily available for use with reduced amounts of physical handling. Moreover, the present invention provides a hard to see intracorneal implant oriented so that the implant can be easily and effectively placed on a surface, e.g., stromal bed, of the eye, in the desired orientation, that is with the anterior face of the implant anterior of the posterior face of the implant, without requiring a visual inspection of the implant. In short, the present invention provides very effective apparatus, systems and methods for packaging/holding intracorneal implants which advantageously reduce the risk of damaging the implants and facilitate the effective use of such implants.

In one broad aspect of the present invention, apparatus for holding intracorneal implants are provided. Such apparatus comprise a holder body having a surface and a through hole extending through the surface. The surface is sized and adapted to carry an intracorneal inlay. The through hole, which extends through the surface, preferably is sized and adapted to facilitate the application of suction to an intracorneal implant located in proximity to the surface. As is discussed in more detail hereinafter, the ability to apply suction to the intracorneal implant is very effective in providing for holding and packaging the implant and for facilitating its use, for example, insertion of the implant into an eye, with a reduced amount of physical handling of or contact with, e.g. touching, the implant. As noted previously, reducing the amount of physical contact with or handling of the implant prior to implantation is effective in reducing the risk that the implant will be harmed or otherwise detrimentally affected.

The through hole preferably extends through substantially the entire length of the holder body. In one embodiment, the through hole includes a first portion located adjacent the surface adapted to carry the implant and a second portion spaced apart from the surface. The first portion preferably has a smaller cross-sectional area than does the second portion. The cross-sectional area of the second portion more preferably is in a range of about two to about six or about eight times as large as the cross-sectional area of the first portion of the through hole. The through hole sized as noted above is effective in facilitating the application of suction to the implant being carried by the surface and in allowing the holder body to be held by an implement passed through at least a portion of the through hole.

Advantageously, the first portion and the second portion of the through hole are co-axial, and more preferably are co-axial with the central axis of the holder body. The first portion and the second portion of the through hole preferably each has a substantially constant cross-sectional area. Still more preferably, the first portion of the through hole abuts, i.e., is directly contiguous with, the second portion of the through hole. However, other configurations of the through hole may be employed and are included within the scope of the present invention. For example, the through hole may have a substantially constant cross-sectional area throughout, may include a tapering cross-section, and the like.

The through hole forms an opening in the surface adapted to carry the implant, which opening is advantageously centrally located on the surface.

The surface of the holder body which is adapted to carry the intracorneal implant preferably has a concave configuration. The surface configuration preferably is at least somewhat consistent with the configuration of the implant as it would be used in a patient's eye. Advantageously, the surface configuration is at least somewhat consistent with the configuration of the anterior face of the implant.

In a very useful embodiment, the holder body further includes a slot formed in the surface adapted to carry the implant and a sidewall of the body and spaced apart from the through hole. Advantageously, the slot extends only across a portion of the surface. In one embodiment, the body includes two of these slots. The through hole preferably is located between two slots. These slots are useful in removing the intracorneal implant from the surface, as is discussed hereinafter.

The holder body preferably includes a first sidewall portion located adjacent the surface adapted to carry the implant and a second sidewall portion spaced apart from the surface. The first sidewall portion preferably has a smaller cross-sectional area than does the second sidewall portion. The first sidewall portion may have any suitable configuration, and preferably has a substantially circular cross-section. The second sidewall portion advantageously includes two substantially oppositely disposed flats. Such flats are effective to facilitate removing the holder body from a container and/or holding the holder body without manually touching the body.

The orientation of the at least one slot, for example, preferably two slots, noted above preferably is substantially parallel to the flats. Such arrangement facilitates holding the holder body while at the same time removing the intracorneal implant from the surface adapted to carry the implant using the slot or slots.

The second sidewall portion preferably includes an end region extending away from the first sidewall portion which has an enlarged cross-sectional area. Such enlarged end region facilitates gripping the holder body, for example at the flats, and reduces the possibility of losing grip of the holder body or of allowing the holder body to fall.

In a further aspect of the present invention, the present apparatus further comprise a cap member sized and adapted to be secured to the holder body so as to hold a intracorneal implant between the surface adapted to carry the implant using and the cap member. In a very useful embodiment, the cap has a through passage forming an opening in a surface of the cap member which partially defines a space sized and adapted to hold an intracorneal implant, that is between the holder body surface adapted to carry an intracorneal implant and the cap member. This through passage in the cap member preferably is sized and adapted to allow liquid, for example, liquid in a container holding the holder body/cap member assembly, to pass there through to contact the intracorneal implant being held in the space between the holder body and the cap member.

In one embodiment, the cap member includes a skirt sized and adapted to engage a sidewall of the holder body to removably secure the cap member to the body. The skirt preferably includes at least one notch, for example, an area of reduced thickness, preferably a through notch, which is effective to provide an effective degree of flexibility or resiliency to the cap so as to provide for effective holding of the cap member to the holder body while, at the same time, allowing for the cap member to be relatively easily removed from the holder body, when such removal is desired.

The present apparatus may further comprise an intracorneal implant, for example, an intracorneal inlay, held between the holder body and the cap member. The intracorneal implant has an anterior face and a posterior face, and preferably is held between the holder body and the cap member so that the anterior face of the implant faces the surface of the holder body sized and adapted to carry the implant. The diameter of the implant preferably is at least about 50% the diameter of this holder body surface. In a still further embodiment, the present apparatus comprises a container with holder body and the cap member being located in the container. The holder body cap member assembly may also include an intracorneal implant located in such assembly placed in the container. The container advantageously also contains a liquid, more preferably an aqueous-based liquid, such as a balanced salt solution, e.g., a commercially available solution, in an amount effective so that at least a portion of the intracorneal implant is in contact with the liquid.

Methods of providing intracorneal implants ready for use are provided and are included within the scope of the present invention. Such methods comprise: providing an intracorneal implant on a surface of a holder body having a through hole extending through the surface; and removing the intracorneal implant from the surface. Apparatus in accordance with the present invention, for example, as described herein, may be, and preferably are, used in practicing the present methods.

In one embodiment, the providing step preferably includes applying suction to the intracorneal implant through the through hole. The suction applied is sufficient or effective to hold the intracorneal implant to the surface of the holder body. In one particularly useful embodiment, the applying step includes squeezing the squeezable bulb of a pipet, passing the stem of the pipet into the through hole and releasing the squeezing pressure, to obtain the desired suction. The pipet should be sized to provide the desired degree of suction. For example, the stem of the pipet preferably is sized to fit substantially snugly in the through hole, while being able to be easily passed in and out of the through hole.

Advantageously, the removing step includes passing the intracorneal implant from the surface of the holder body directly to a surface, for example, the stromal bed, of an eye. A manually operated instrument, such as a spatula, forceps and the like, can be placed in the slot or slots of the holder body to urge the implant away from the holder body surface. This “direct passing” approach is particularly useful since handling or contact with (touching) the implant is reduced, or even substantially minimized or eliminated. In addition, this approach advantageously places the implant on the surface of the eye in the proper orientation, that is with the anterior face of the implant located anterior of the posterior face of the implant.

In one embodiment, the removing step may include manually removing the intracorneal implant from the surface, for example, by using one or more manually operated instruments, such as tweezers, forceps and the like. The removing step may include gripping or grasping the intracorneal implant, for example, on the surface of the holder body, with a forceps. The removed intracorneal implant can then be very easily placed or positioned for placement in the eye of a patient, as desired.

In any event, the present methods preferably further comprise placing the intracorneal implant in an eye.

Methods for packaging intracorneal implants are also provided and are included within the scope of the present invention. Such methods comprise placing an intracorneal implant on a surface of a holder body which has a through hole extending through the surface, preferably with the anterior face of the implant facing the holder body surface; and securing a cap member to the holder body so as to hold the intracorneal implant between the surface and the cap, preferably so that the anterior face of the implant is maintained facing the holder body surface. The resulting package assembly very effectively protects the intracorneal implant and is very effective in providing the implant ready for use by the surgeon. The holder body and cap member used in these packaging methods can be, and preferably are, as described herein with regard to other embodiments of the present invention.

The present packaging methods preferably further comprise placing the package assembly, that is the holder body, implant and cap member secured to the holder body, in a container. More preferably, the present packaging methods further comprise placing a liquid, for example, an aqueous-based liquid, e.g., saline, balanced salt solution and the like, in the container.

Each and every feature described herein, and each and every combination of two or more of such features, is included within the scope of the present invention provided that the features included in such a combination are not mutually inconsistent.

These and other aspects and advantages of the present invention are set forth in the following detailed description and claims, particularly when considered in conjunction with the accompanying drawings in which like parts bear alike reference numerals.

### BRIEF DESCRIPTION OF THE DRAWINGS

FIG. 1 is a schematic, exploded view showing an embodiment of the present apparatus.

FIG. 2 is a perspective, exploded view of certain components of the embodiment of the present invention shown in FIG. 1.

FIG. 3 is a cross-sectional view of the embodiment shown in FIG. 2 with the parts assembled.

FIG. 4 is a schematic view showing the assembled embodiment of FIG. 3 placed in a container filled with aqueous-based liquid.

FIG. 5 is a schematic view showing the assembled holder of FIG. 3 being removed from the liquid filled container.

FIG. 6 is a schematic view showing the assembly removed from the container using a pipet.

FIG. 7 is a schematic view showing the holder body of the embodiment in FIG. 6 with the intracorneal implant in position to be separated from the holder body.

### DETAILED DESCRIPTION OF THE INVENTION

Referring now to the drawings, FIGS. 1, 2 and 3 illustrate an embodiment of the apparatus of the present invention. This apparatus, shown generally at 10, includes a package insert 12, a cap insert 14, an intracorneal inlay 16, and a pipet 18. The intracorneal inlay 16 includes an anterior face 15 and a posterior face 17 and is made of any suitable material effective as an intracorneal inlay and useful and compatible with a living cornea. Such implant materials of construction include, but are not limited to, hydrogels, collagen, collagen/hydrogel composites and the like. A particular example of an intracorneal inlay which may be used in accordance with the present invention is an inlay commonly known as a Kougar Inlay. In addition, it should be noted that the intracorneal inlay 16 can be replaced by other suitable intracorneal structures, for example, intracorneal onlays, other intracorneal implants and the like.

In the particular embodiment illustrated, intracorneal inlay 16 comprises a synthetic hydrogel which is to be hydrated when implanted into an eye.

Pipet 18 is of conventional construction and includes a hollow stem 20 having a distal tip region 22 of reduced cross-sectional area relative to the cross-sectional area of the proximal portion of stem 20. Pipet 18 further includes a proximately located squeezable bulb 24 which is in fluid communication with hollow stem 20. By activating bulb 24, for example, by applying squeezing pressure to bulb 24 and then releasing the squeezing pressure, reduced pressure or partial vacuum is formed in the hollow stem 20 with the distal end 26 of stem 18 being closed. Put another way, squeezing and releasing the squeezable bulb 24 creates suction at the distal tip 26 of hollow stem 20.

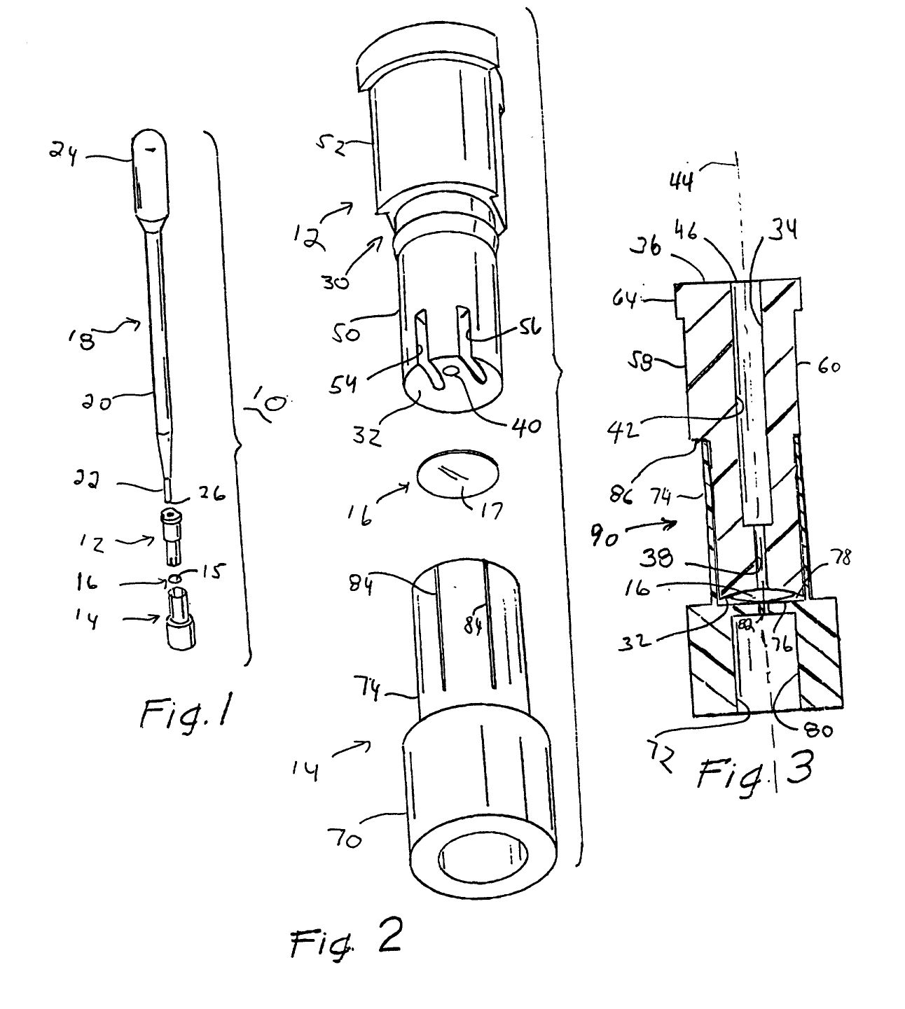

FIGS. 2 and 3 show package insert 12, intracorneal inlay 16 and cap insert 14 in more detail.

Package insert 12 includes a holder body 30 having a surface 32 sized, configured and adapted to carry intracorneal inlay 16. Surface 32 has a generally concave configuration which is consistent with or complimentary to the generally convex surface of anterior face 15 of inlay 16.

A through hole 34 is located through the entire length of the body 30, that is from the end surface 36 to the surface 32 which is located at the opposing end of the body 30. Through hole 34 includes a first portion 38 which forms a centrally located opening 40 in surface 32. A second portion 42 of through hole 34 is positioned away from the surface 32 and extends along the central axis 44 of holder body 30 and forms an opening 46 in end surface 36. First portion 38 and second portion 42 of through hole 34 are coaxial and extend substantially along the central axis 44 of holder body 30. The first portion 38 of through hole 34 has a substantially constant cross-sectional area. Second portion 42 of through hole 34 also has a substantially constant cross-sectional area. Both portions 38 and 42 have substantially circular cross-sections, although other configurations may be used. The cross-sectional area of second portion 42 is at least about 2 times, preferably about 2.5 to about 6 or about 8 times, as large as the cross-sectional area of the first portion 30 to about 3 times as large as the diameter of first portion 38, both portions having a substantially circular cross-section.

In addition, holder body 30 includes a first sidewall portion 50 which has a substantially circular cross-section and a second sidewall portion 52 which has a somewhat larger cross-section than does first sidewall portion 50.

Slots 54 and 56 are positioned on either side of through hole 34 and opening 40. Slots 54 and 56 extend part way up first sidewall portion 50 and part way across surface 32.

Second sidewall portion 52 includes two substantially opposing flats 58 and 60. Slots 54 and 56 are oriented substantially parallel to the length dimension of flats 58 and 60. In addition, second sidewall portion 52 includes an enlarged end portion 64 adjacent end 36.

Cap insert 14 includes an enlarged end region 70 which defines an internal passage 72, and a skirt 74 which extends away from passage 72. Cap insert 14 includes a surface 76 which partially defines the space 78 in which the intracorneal inlay 16 is located when the package insert 12 and cap insert 14 are assembled together, as shown in FIG. 3. Internal passage 72 includes an enlarged portion 80 and a portion 82, having a substantially smaller cross-section than does portion 80, which forms an opening in the surface 76.

The skirt 74 of cap insert 14 includes a series of longitudinally extending through notches 84 which are spaced around skirt 74. Such notches 84 are effective to provide a desired degree of flexibility and resilience to skirt 74. To allow the cap insert 14 to be effectively secured to package insert 12 and to allow the cap insert to be easily removed from the package insert, when such removed is desired.

Skirt 74 is sized and adapted to receive and hold the first sidewall portion 50 of package insert 12, as shown in FIG. 3. Both the package insert 12 and cap insert 14 are sized so that the skirt 74 can be placed to substantially abut surface 86 of second sidewall portion 52, again as shown in FIG. 3. This arrangement allows space 78 to be maintained between surface 32 and surface 76.

The package insert 12 and the cap insert 14 may be made of any suitable material or materials of construction. Such materials should be effective to perform in accordance with the present invention and should have no significant detrimental effect on the intracorneal implant being packaged using such inserts. In one very useful embodiment both the package insert 12 and the cap insert 14 are made of the same material. Very useful materials of construction include polymeric materials. If the package insert 12 and cap insert 14 are to be subjected to elevated temperatures, for example, for sterilization, useful materials of construction include, but are not limited to, polysulfone, polyurethane and the like. In the event that the inserts are to be subjected to no elevated temperatures, suitable materials of construction include, but are not limited to, polyolefins, such as polypropylene and the like, polycarbonates and the like.

The size of the package insert 12 and the cap insert 14 may vary. these two components should be of sizes so that each of the components is compatible with the other and the intracorneal inlay 16 in accordance with the present invention. The maximum diameter of each of the package insert 12 and the cap insert 14 preferably is in the range of about 0.2 inches to about 0.5 or about 0.6 inches. Each of these inserts preferably have lengths ranging from about 0.4 inches to about 2 inches. The size of these inserts should be chosen to provide for adequate protection and hydration of the intracorneal implant being packaged and to facilitate their use in assisting implanting the implant into an eye. If the inserts 12 and 14 are too small, they will not adequately protect the implant being packaged. If the inserts are too large, the intracorneal implant may move and become misoriented in space 78, and/or it may be difficult to draw the proper degree of suction to hold the implant to the package insert and/or to use the package insert to facilitate implanting the intracorneal implant.

Preferably the surface 32 is sized so that the intracorneal inlay 16 is at least about 50%, more preferably at least about 70%, as large as surface 32. Such sizing reduces the need to visually locate the inlay 16, which is often hard to see with the naked eye, on the surface 32 prior to removing the inlay from this surface.

The combination of the package insert 12 and the cap insert 14 is effective for use in packaging intracorneal inlay 16. In one embodiment, the intracorneal inlay 16 is placed on the concave surface 32 of package insert 12, preferably so that the anterior face 15 of the inlay is facing surface 32. This can be done, for example, by transferring the inlay 16 to the concave surface 32 by first placing the inlay 16 on a convex surface, preferably so that the posterior face 17 of the inlay 16 is facing the convex surface, and then transferring the inlay to surface 32. A limited degree of suction can be applied to the inlay 16 to cause the inlay to adhere to the surface 32. Such suction can be obtained by placing the stem 20 of pipet 18 into through hole 34 and releasing the squeeze bulb 24 to cause suction to be applied to inlay 16.

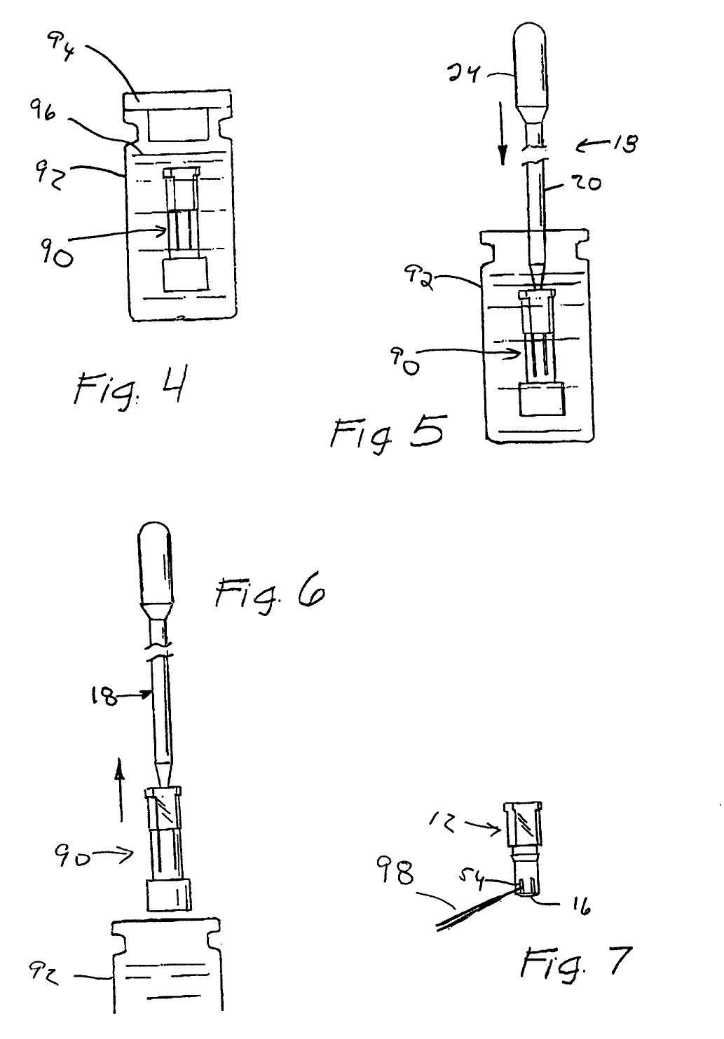

Once the inlay 16 is in contact with the concave surface 32 of package insert 12, cap insert 14 can be secured to the package insert by passing the first sidewall portion 50 of the package insert into the space defined by skirt 74, thereby securing package insert 12 to cap insert 14. Once this securement has occurred, the package assembly shown generally at 90 in FIG. 3 is placed in a container 92, see FIG. 4. The package assembly 90 is placed in container 92 so that the end surface 36 is located nearest the top of the container. A quantity of aqueous-based liquid, for example, buffered saline or balanced salt solution 96 is provided to substantially immerse the package assembly 90. This container 92, closed with container cap 94 and including the package assembly 90, can then be transported and stored.

When it is determined that the intracorneal inlay 16 is to be implanted into an eye, the cap 94 of the container 92 is removed and the stem 20 of pipet 18 is introduced into through hole 34. Squeezing pressure on squeeze bulb 24 is released so that a limited amount of suction is applied to inlay 16 in space 78. Using the pipet 18, the package assembly 90 is removed from container 92 as shown is FIG. 6. In an alternate embodiment, the package assembly 90 can be removed from container 92 using a pair of gripping scissors as other gripping means placed on the opposing flats 58 and 60. The enlarged end portion 64 is effective to reduce the risk that the package insert 90 will slip.

The cap insert 14 is removed from the package insert 12 and the pipet 18 is removed from the package insert. The inlay 16 remains held by the package insert in contact with surface 32. At this point, the inlay is ready to be implanted into the eye.

In one embodiment, as generally shown in FIG. 7, the inlay 16, still in contact with the package insert 12, can be moved in position for implantation into the eye. For example, the package insert 12 can be placed in intimate contact with the surface, e.g., stromal bed, of the eye. If needed, a spatula 98 is placed into slot 54 to facilitate separation of the inlay 16 from the package insert 12. Very little, if any, touching of the inlay 16 is needed to achieve this separation. The package insert 12 is then withdrawn from the surface of the eye. The inlay 16 remains on the surface of the eye, properly oriented so that the anterior face 15 of inlay 16 is anterior of posterior face 17. Moreover, the package insert 12, and particularly the surface 32, is sufficiently small in size so that the inlay 16 is placed at substantially the proper location on the surface of the eye. Once the inlay is located on the stromal bed, the surgeon, now using visual magnification (a microscope) can adjust the position of the inlay 16 and complete the inlay implantation procedure.

Alternately, a forceps is used to remove the inlay 16 from the package insert 12. This is done by passing one portion of the forceps 78 into slot 54 (or slot 56) and grasping the inlay 16 with the other portion of the forceps 98. The inlay 16 can then easily be removed from the package insert 12 and placed in position for implantation in an eye.

While this invention has been described with respect to various specific examples and embodiments, it is to be understood that the invention is not limited thereto and that it can be variously practiced within the scope of the following claims:

What is claimed is:

1. An apparatus for holding an intracorneal implant comprising:
a holder body having a surface sized and adapted to carry an intracorneal implant, and a through hole extending through the surface.

2. The apparatus of claim 1 wherein the through hole is sized and adapted to facilitate suction being applied to an intracorneal implant located in proximity to the surface.

3. The apparatus of claim 1 wherein the holder body has a length and the through hole extends through substantially the entire length of the body.

4. The apparatus of claim 1 wherein the through hole includes a first portion located adjacent the surface and a second portion spaced apart from the surface, the first portion having a smaller cross-sectional area than the second portion.

5. The apparatus of claim 4 wherein the cross-sectional area of the second portion is in a range of about 2 to about 6 times as large as the cross-sectional area of the first portion.

6. The apparatus of claim 4 wherein the first portion and the second portion are co-axial.

7. The apparatus of claim 4 wherein at least one of the first portion and the second portion has a substantially constant cross-sectional area.

8. The apparatus of claim 4 wherein the first portion abuts the second portion.

9. The apparatus of claim 1 wherein the through hole forms a substantially centrally located opening in the surface.

10. The apparatus of claim 1 wherein the surface has a concave configuration.

11. The apparatus of claim 1 wherein the holder body further includes a slot formed in the surface and a sidewall of the holder body and being spaced apart from the through hole.

12. The apparatus of claim 11 wherein the slot extends across only a portion of the surface.

13. The apparatus of claim 11 wherein the holder body includes two of the slots.

14. The apparatus of claim 13 wherein the through hole is located between the two slots.

15. The apparatus of claim 1 wherein the holder body includes a first sidewall portion located adjacent the surface and a second sidewall portion spaced apart from the surface.

16. The apparatus of claim 15 wherein the first sidewall portion has a smaller cross-sectional area than the second sidewall portion.

17. The apparatus of claim 15 wherein the first sidewall portion has a substantially circular cross-section.

18. The apparatus of claim 15 wherein the second sidewall portion includes two substantially oppositely disposed flats.

19. The apparatus of claim 18 wherein the holder body further includes at least one slot formed in the surface and the first sidewall portion and spaced apart from the through hole, the at least one slot being oriented substantially parallel to the flats.

20. The apparatus of claim 15 wherein the second sidewall portion includes an end region extending away from the first sidewall portion having an enlarged cross-sectional area.

21. The apparatus of claim 1 which further comprises a cap member sized and adapted to be secured to the holder body so as to hold an intracorneal implant between the surface and the cap member.

22. The apparatus of claim 21 wherein the cap member has a through passage forming an opening in a surface of the cap member partially defining a space sized and adapted to hold an intracorneal implant.

23. The apparatus of claim 22 wherein the through passage is sized and adapted to allow liquid to pass therethrough to contact an intracorneal implant held in the space.

24. The apparatus of claim 21 wherein the cap member includes a skirt sized and adapted to engage a sidewall of the holder body to removably secure the cap member to the holder body.

25. The apparatus of claim 24 therein the skirt includes at least one notch.

26. The apparatus of claim 21 which further comprises an intracorneal implant held between the holder body and the cap member.

27. The apparatus of claim 21 which further comprises a container, with the holder body and the cap member being located in the container.

28. The apparatus of claim 26 which further comprises a container, with the holder body, the cap member and the intracorneal implant being located in the container.

29. The apparatus of claim 28 which further comprises a liquid located in the container in an amount effective so that at least a portion of the intracorneal implant is in contact with the liquid.

30. The apparatus of claim 29 where the liquid is an aqueous-based liquid.

31. A method of providing an intracorneal implant ready for use comprising:
providing an intracorneal implant in contact with a surface of a holder body having a through hole extending through the surface; and
removing the intracorneal inlay from the surface.

32. The method of claim 31 wherein the providing step includes:
applying suction to the intracorneal inlay, the suction being effective to hold the intracorneal inlay in contact with the surface.

33. The method of claim 32 wherein the applying step includes passing a stem of a pipet into the through hole and releasing a squeezable bulb of the pipet to obtain the desired suction.

34. The method of claim 31 wherein the removing step includes passing the intracorneal implant from the surface directly to a surface of an eye.

35. The method of claim 31 wherein the removing step includes using a manually operated instrument.

36. The method of claim 31 wherein the removing step includes touching the intracorneal implant with a manually operated instrument.

37. The method of claim 31 which further comprises placing the intracorneal implant in an eye.

38. A method of packaging an intracorneal implant comprising:
placing an intracorneal implant in proximity to a surface of a holder body which has a through hole extending through the surface; and
securing a cap member to the holder body so as to hold the intracorneal lens between the surface and the cap member, thereby forming a package assembly.

39. The method of claim 38 wherein the cap member has a through passage sized and adapted to allow liquid to pass therethrough to contact the held intracorneal implant.

40. The method of claim 38 which further comprises placing the package assembly in a container.

41. The method of claim 40 which further comprises placing a liquid in the container.

42. The method of claim 41 wherein the liquid is a aqueous-based liquid.
